(19) United States
(12) Patent Application Publication
(10) Pub. No.: US 2011/0059019 A1
(43) Pub. Date: Mar. 10, 2011

(54) NOVEL ARYL PIPERAZINE DERIVATIVES USEFUL AS MODULATORS OF DOPAMINE AND SEROTONIN RECEPTORS

(75) Inventors: Dan Peters, Malmo (SE); Lars Christian Rønn, Hillerod (DK); Karin Sandager Nielsen, Fredensborg (DK); Jørgen Scheel-Krüger, Glostrup (DK)

(73) Assignee: NeuroSearch A/S, Ballerup (DK)

(21) Appl. No.: 12/865,410

(22) PCT Filed: Jan. 29, 2009
(86) PCT No.: PCT/EP09/50999

§ 371 Date: Nov. 26, 2010

Related U.S. Application Data
(60) Provisional application No. 61/025,906, filed on Feb. 4, 2008.

(30) Foreign Application Priority Data
Feb. 1, 2008 (DK) PA 2008 00149

(51) Int. Cl. A61K 31/496; C07D 401/14; A61K 49/00; A61P 25/00; A61P 25/14; A61P 29/00; A61P 15/00; A61P 25/30; A61P 25/22; A61P 25/18

(52) U.S. Cl. 424/9.1; 544/363; 514/253.06

(57) ABSTRACT

This invention provides novel aryl piperazine derivatives having medical utility, in particular as modulators of dopamine and serotonin receptors, preferably the D₃, 5HT_{1A} and 5-HT_{2A} receptor subtypes, and in particular useful for the treatment of neuropsychiatric disorders, incl. schizophrenia.

### TECHNICAL FIELD

This invention provides novel aryl piperazine derivatives having medical utility, in particular as modulators of dopamine and serotonin receptors, preferably the D₃, 5HT_{1A} and 5-HT_{2A} receptor subtypes, and in particular useful for the treatment of neuropsychiatric disorders, incl. schizophrenia.

### BACKGROUND ART

Dopamine is involved in several important functions, excitatory and inhibitory, via dopaminergic receptors in the central and peripherical nervous system. Dopamine receptors were originally classified into two main groups: D₁ and D₂. The five currently cloned dopamine receptors fall into these classes. Thus, the D₁-like receptors include D₁ and D₅, while the D₂-like receptors include D₂, D₃ and D₄.

The dopamine receptors, and in particular the D₂-like receptors, are recognised as potential therapeutic targets for various neurological and psychiatric disorders, in particular psychotic disorders, incl. schizophrenia. Other therapeutic indications associated with the dopamine receptors include depression, Parkinson's disease, Huntington's disease, movement disorders such as dystonia, anxiety, restlessness, obsessive-compulsive disorders, mania, geriatric disorders, dementia, sexual dysfunction, musculo-skeletal pain symptoms, e.g. pain associated with fibromyalgia, substance abuse (cocaine abuse and addiction), abuse liability and withdrawal symptoms in drug addicts, and sleep disorders.

Still other therapeutic indications include eating disorders such as overeating, compulsive overeating, inability to regulate eating, bulimia and Binge-eating disorder.

Also the compounds of the invention may be useful for the treatment of abuse liability and withdrawal symptoms caused by termination of use of addictive substances. Such addictive substances include nicotine containing products such as tobacco, opioids such as heroin, cocaine and morphine, cannabis, benzodiazepines, benzodiazepine-like drugs, and alcohol. Withdrawal from addictive substances is in general a traumatic experience characterised by anxiety and frustration, anger, anxiety, difficulties in concentrating, restlessness, decreased heart rate and increased appetite and weight gain.

Finally receptor selective ligands find use as diagnostic tools in diagnostic methods, and in particular for in vivo receptor imaging (neuroimaging).

WO 2006/072608 describes aryl piperazine derivatives useful as as modulators of dopamine and serotonin receptors. However, the aryl piperazine derivatives of the present invention have not been reported.

### SUMMARY OF THE INVENTION

According to the present invention it has now been found that a particular group of aryl piperazine derivatives show superior activity as modulators of dopamine and serotonin receptors, preferably the D₃, 5HT_{1A} and 5-HT_{2A} receptor subtypes, has no significant activity on hERG, and has a good bioavailability when administered p.o.

Therefore, in its first aspect, the invention provides novel aryl piperazine derivatives represented by Formula I

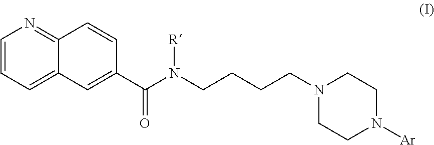

a stereoisomer thereof or a mixture of its stereoisomers, or a pharmaceutically acceptable salt thereof, wherein

R′ represents hydrogen or alkyl; and

Ar represents naphthyl or a heterocyclic, monocyclic or bicyclic aromatic group, which aromatic groups may optionally be substituted one or more times with substituents selected from alkyl, alkoxy, halo, trifluoromethyl, nitro and cyano.

In another aspect the invention relates to the use of the aryl piperazine derivative of the invention, or a pharmaceutically acceptable salt thereof, or a prodrug thereof for the manufacture of a pharmaceutical composition.

Viewed from yet another aspect the invention relates to the use of the aryl piperazine derivative of the invention, or a pharmaceutically acceptable salt thereof, or a prodrug thereof, for use as a medicament, or for the manufacture of a pharmaceutical composition for the treatment, prevention or alleviation of a disease or a disorder or a condition of a mammal, including a human, which disease, disorder or condition is responsive to modulation of the dopamine and serotonin receptors.

In a final aspect the invention provides a method of diagnosis, treatment, prevention or alleviation of a disease or a disorder or a condition of a living animal body, including a human, which disorder, disease or condition is responsive to modulation of the dopamine and serotonin receptors, in particular the D₃, D₂-like and 5-HT₂ receptor subtypes, preferably the dopamine D₃ receptor subtype and/or the D₃/5-HT_{1A} or D₃/5-HT_{2A} receptor sybtypes, which method comprises the step of administering to such a living animal body in need thereof, a therapeutically effective amount of the aryl piperazine derivative of the invention, or a pharmaceutically acceptable salt thereof, or a prodrug thereof.

Other objects of the invention will be apparent to the person skilled in the art from the following detailed description and examples.

### DETAILED DISCLOSURE OF THE INVENTION

According to the present invention it has now been found that a particular group of aryl piperazine derivatives show a superior biological profile as modulators of dopamine and serotonin receptors.

Therefore, in its first aspect, the invention provides novel aryl piperazine derivatives represented by Formula I

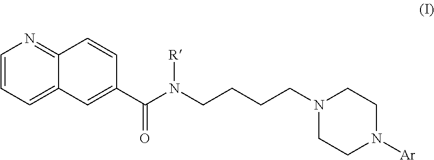

a stereoisomer thereof or a mixture of its stereoisomers, or a pharmaceutically acceptable salt thereof, wherein

R′ represents hydrogen or alkyl; and

Ar represents naphthyl or a heterocyclic, monocyclic or bicyclic aromatic group, which aromatic groups may optionally be substituted one or more times with substituents selected from alkyl, alkoxy, halo, trifluoromethyl, nitro and cyano.

In a preferred embodiment the aryl piperazine derivative of the invention is a compound of Formula I, or a pharmaceutically acceptable salt thereof, wherein R′ represents hydrogen or alkyl.

In a more preferred embodiment R′ represents hydrogen.

In another more preferred embodiment R′ represents alkyl, and in particular methyl.

In another preferred embodiment the aryl piperazine derivative of the invention is a compound of Formula I, or a pharmaceutically acceptable salt thereof, wherein Ar represents naphthyl or a heterocyclic, monocyclic or bicyclic aromatic group, which aromatic groups may optionally be substituted one or more times with substituents selected from alkyl, alkoxy, halo, trifluoromethyl, nitro and cyano.

In a more preferred embodiment Ar represents naphthyl or a heterocyclic, monocyclic or bicyclic aromatic group, which aromatic groups may optionally be substituted one or more times with substituents selected from alkyl, alkoxy, halo, trifluoromethyl, nitro and cyano.

In an even more preferred embodiment Ar represents naphthyl, optionally substituted once or twice with substituents selected from alkyl, alkoxy, halo, trifluoromethyl, nitro and cyano.

In a still more preferred embodiment Ar represents naphthyl, and in particular naphth-1-yl or naphth-2-yl.

In another more preferred embodiment Ar represents a heterocyclic, monocyclic or bicyclic aromatic group, which aromatic groups may optionally be substituted once or twice with substituents selected from alkyl, alkoxy, halo, trifluoromethyl, nitro and cyano.

In an even more preferred embodiment Ar represents a heterocyclic, monocyclic or bicyclic aromatic group selected from thiazolyl, pyridinyl, pyridazinyl, pyrimidinyl and isoquinolinyl, which aromatic group may optionally be substituted once or twice with substituents selected from alkyl, alkoxy, halo, trifluoromethyl, nitro and cyano.

In a still more preferred embodiment Ar represents thiazolyl, and in particular thiazol-2-yl.

In another still more preferred embodiment Ar represents pyridinyl, optionally substituted once or twice with substituents selected from alkyl, alkoxy, halo, trifluoromethyl, nitro and cyano.

In a yet more preferred embodiment Ar represents pyridinyl, optionally substituted one or two times with alkyl, and in particular methyl.

In another yet more preferred embodiment Ar represents pyridinyl, optionally substituted with alkyl, and in particular methyl.

In a third yet more preferred embodiment Ar represents pyridinyl substituted with alkyl, and in particular methyl.

In a fourth yet more preferred embodiment Ar represents pyridinyl, and in particular pyridin-2-yl or pyridin-4-yl.

In a third more preferred embodiment Ar represents pyridazinyl, and in particular pyridazin-3-yl.

In a fourth more preferred embodiment Ar represents pyrimidinyl, and in particular pyrimidin-2-yl.

In a fifth more preferred embodiment Ar represents isoquinolinyl, and in particular isoquinolin-1-yl.

In a most preferred embodiment the aryl piperazine derivative of the invention is

Quinoline-6-carboxylic acid [4-(4-pyridin-2-yl-piperazin-1-yl)-butyl]-amide; or

Quinoline-6-carboxylic acid {4-[4-(6-methyl-pyridin-2-yl)-piperazin-1-yl]-butyl}-amide;

a stereoisomer thereof or a mixture of its stereoisomers, or a pharmaceutically acceptable salt thereof.

Any combination of two or more of the embodiments described herein is considered within the scope of the present invention.

### Substituents

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, hexyl and isohexyl. In a preferred embodiment alkyl represents a C₁₋₄-alkyl group, including butyl, isobutyl, secondary butyl, and ertiary butyl. In another preferred embodiment of this invention alkyl represents a C₁₋₃-alkyl group, which may in particular be methyl, ethyl, propyl or isopropyl.

In the context of this invention an alkoxy group designates an “alkyl-O-” group, wherein alkyl is as defined above. Examples of preferred alkoxy groups of the invention include methoxy, ethoxy and isopropoxy.

In the context of this invention halo represents fluoro, chloro, bromo or iodo.

### Steric Isomers

It will be appreciated by those skilled in the art that the compounds of the present invention may exist in different stereoisomeric forms, including enantiomers, diastereomers, as well as geometric isomers (cis-trans isomers). The invention includes all such stereoisomers and any mixtures thereof including racemic mixtures.

Racemic forms can be resolved into the optical antipodes by known methods and techniques. One way of separating the enantiomeric compounds (including enantiomeric intermediates) is—in the case the compound being a chiral acid—by use of an optically active amine, and liberating the diastereomeric, resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L-(tartrates, mandelates, or camphorsulphonate) salts for example.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optically active starting materials or intermediates.

### Pharmaceutically Acceptable Salts

The aryl piperazine derivatives of the invention may be provided in any form suitable for the intended administration. Suitable forms include pharmaceutically (i.e. physiologically) acceptable salts, and pre- or prodrug forms of the aryl piperazine derivatives of the invention.

Examples of pharmaceutically acceptable salts include, without limitation, the non-toxic inorganic and organic acid salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

### Steric Isomers

Some of the aryl piperazine derivatives of the present invention may exist in (+) and (−) forms as well as in racemic forms (±). The racemates of these isomers and the individual isomers themselves are within the scope of the present invention.

Racemic forms can be resolved into the optical antipodes by known methods and techniques. One way of separating the diastereomeric salts is by use of an optically active acid, and liberating the optically active amine compound by treatment with a base. Another method for resolving racemates into the optical antipodes is based upon chromatography on an optical active matrix. A stereo-selective synthetic approach may be pursued. Racemic compounds of the present invention can thus be resolved into their optical antipodes, e.g., by fractional crystallisation of D- or L-(tartrates, mandelates or camphorsulphonate) salts for example.

Starting materials and/or intermediate compounds used for producing the chemical compounds of the present invention may also be resolved by the formation of diastereomeric amides by reaction of the aryl piperazine derivative of the present invention with an optically active activated carboxylic acid such as that derived from (+) or (−) phenylalanine, (+) or (−) phenylglycine, (+) or (−) camphanic acid or by the formation of diastereomeric carbamates by reaction of the starting material or intermediate compound for use according to the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials.

### Methods of Preparation

The aryl piperazine derivatives of the invention may be prepared by conventional methods for chemical synthesis, e.g. those described in the working examples.

Generally amides may be prepared by transforming acids or acid chlorides into the corresponding hydroxy amides by a standard procedure. Esters may be obtained by reacting acidic starting materials with 1,4-dihydroxybutane. After substitution of the terminal hydroxy group by bromine, hydroxyl amides may be treated with the aryl piperazine in the presence of a base to give the desired end product. Compounds based on a ethereal tether may be synthesized starting from the appropriate phenol, which is then condensed with 14-dihydroxybutane or 1,5-dihydroxypentane, followed by transformation into the final products as described above.

Intermediate compounds invention may be resolved by the formation of diastereomeric amides by reaction with an optically active activated carboxylic acid such as that derived from (+) or (−) phenylalanine, (+) or (−) phenylglycine, (+) or (−) camphanic acid or by the formation of diastereomeric carbamates by reaction of the intermediate compound with an optically active chloroformate or the like.

### Biological Activity

The aryl piperazine derivatives of the invention were found to possess selectivity for the dopamine and serotonin receptors. Therefore, in a preferred embodiment, the invention relates to use of the aryl piperazine derivatives of the invention for the treatment, prevention or alleviation of a disease or a disorder or a condition of a mammal, including a human, which disease, disorder or condition is responsive to modulation of the dopamine and serotonin receptors, in particular the D₃, 5HT_{1A} and 5-HT_{2A} receptor subtypes.

Moreover, the aryl piperazine derivatives of the invention has no significant activity on hERG, and has a good bioavailability when administered p.o.

Therefore, in a preferred embodiment, the invention relates to use of the aryl piperazine derivatives of the invention for the treatment, prevention or alleviation of a disease or a disorder or a condition of a mammal, including a human, which disease, disorder or condition is responsive to modulation of the dopamine and serotonin receptors.

In a more preferred embodiment the disease, disorder or condition is a neurological or psychiatric disorders, in particular psychotic disorders, incl.

schizophrenia, depression, Parkinson's disease, Huntington's disease, movement disorders, in particular dystonia, anxiety, restlessness, obsessive-compulsive disorders, mania, geriatric disorders, dementia, sexual dysfunction, musculo-skeletal pain symptoms, in particular pain associated with fibromyalgia, sleep disorders, substance abuse or addiction, and abuse liability and withdrawal symptoms in drug addicts, cocaine abuse or addiction.

In an even more preferred embodiment the disease, disorder or condition is a neurological or psychiatric disorder, in particular a psychotic disorder, preferably schizophrenia.

In another preferred embodiment the disease, disorder or condition contemplated according to the invention is schizophrenia or Parkinson's disease.

In a third preferred embodiment the disease, disorder or condition contemplated according to the invention an eating disorder, overeating, compulsive overeating, inability to regulate eating, bulimia or Binge-eating disorder.

In a fourth preferred embodiment the disease, disorder or condition contemplated according to the invention is abuse liability or withdrawal symptoms caused by termination of use of addictive substances. Such addictive substances include nicotine containing products such as tobacco, opioids such as heroin, cocaine and morphine, cannabis, benzodiazepines, benzodiazepine-like drugs, and alcohol. Withdrawal from addictive substances is in general a traumatic experience characterised by anxiety and frustration, anger, anxiety, difficulties in concentrating, restlessness, decreased heart rate and increased appetite and weight gain.

In yet another preferred embodiment the aryl piperazine derivatives of the invention are used as diagnostic tools in diagnostic methods, and in particular for in vivo receptor imaging (neuroimaging).

### Pharmaceutical Compositions

In another aspect the invention provides novel pharmaceutical compositions comprising a therapeutically effective amount of the aryl piperazine derivative of the invention.

While an aryl piperazine derivative of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the aryl piperazine derivative of the invention, or a pharmaceutically acceptable salt or derivative thereof, together with one or more pharmaceutically acceptable carriers, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be prepared by any person skilled in the art, by use of standard methods and conventional techniques, appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

In another aspect the invention provides a method for the diagnosis, treatment, prevention or alleviation of a disease or a disorder or a condition of a living animal body, including a human, which disease, disorder or condition is responsive to modulation of the dopamine and serotonin receptors, and which method comprises administering to such a living animal body, including a human, in need thereof an effective amount of an aryl piperazine derivative of the invention.

In the context of this invention the term “treatment” covers treatment, prevention, prophylaxis or alleviation, and the term “disease” covers illnesses, diseases, disorders and conditions related to the disease in question.

The preferred indications contemplated according to the invention are those stated above.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

### EXAMPLES

The invention is further illustrated with reference to the following examples, which are not intended to be in any way limiting to the scope of the invention as claimed.

### Example 1

### Preparatory Example

6-Quinolinecarboxylic acid (Intermediate Compound)

To a solution of 6-methylquinoline (100.0 mg, 0.70 mmol) in H₂O (1.0 mL) and H₂SO₄ (0.25 mL) chromium trioxide (272.0 mg, 2.72 mmol) was added in portions at 0° C. and refluxing for twenty-four hours. The crystalline precipitate of the hydrosulphate which separated upon cooling was removed by filtration, dissolved in 10% sodium hydroxide water solution and, after wash with hexane, was re-precipitated with acetic acid to give 85.0 mg of title compound (70% yield) that was used in the following step without further purification.

¹H NMR, 300 MHz, (DMSO-_{d6}) δ 7.61 (dd, 1H, J₁=8.3 Hz, J₂=4.2 Hz), 8.08 (d, 1H, J=8.8 Hz), 8.20 (dd, 1H, J₁=8.8 Hz, J₂=1.7 Hz), 8.56 (d, 1H, J=8.2 Hz), 8.67 (m, 1H), 9.00 (dd, 1H, J₁=4.1 Hz, J₂=1.5 Hz), 13.20 (br s, 1H); ¹³C NMR, 300 MHz, (DMSO-_{d6}) 122.9, 127.9, 129.2, 129.5, 130.0, 131.7, 138.2, 150.0, 153.4, 167.7; ESI-MS m/z 196 [M+Na]⁺, 174 [M+H]⁺. Anal. (C₁₀H₇NO₂) C, H, N.

N-(4-Hydroxybutyl)quinoline-6-carboxamide (Intermediate Compound)

To a solution of 6-quinolinecarboxylic acid (200.0 mg, 1.16 mmol) in dry dichloromethane (20.0 mL), triethylamine (162.0 μL, 1.16 mmol), 1-hydroxybenzotriazole hydrate (171.0 mg, 1.27 mmol) and N-(3-dimethylaminopropyl)-N′-ethylcarbodiimide hydrochloride (243.0 mg, 1.27 mmol) were added at 0° C. under argon atmosphere; the suspension was warmed to room temperature and stirred for 1 h. Then 4-amino-1-butanol (117.0 μL, 1.27 mmol) was added and the mixture was stirred overnight at room temperature. The resulting suspension was evaporated and the crude product was purified by means of flash chromatography (10% methanol in chloroform) to give 275.0 mg of the title compound as a white solid (97% yield).

Mp (methanol) 121-122° C.; ¹H NMR, 300 MHz, (CDCl₃) δ 1.67-1.84 (m, 4H), 2.13 (br s, 1H); 3.48-3.59 (m, 2H), 3.76 (m, 2H), 7.02 (br s, 1H), 7.43 (m, 1H), 8.01-8.12 (m, 2H), 8.20 (d, 1H, J=8.5 Hz), 8.30 (m, 1H), 8.94 (m, 1H). ESI-MS m/z 511 [2M+Na]⁺, 267 [M+Na]⁺, 245 [M+H]⁺. Anal. (C₁₄H₁₆N₂O₂) C, H, N.

N-(4-Bromobutyl)quinoline-6-carboxamide (Intermediate Compound)

To a solution of N-(4-hydroxybutyl)quinoline-6-carboxamide (500.0 mg, 2.05 mmol) in dry acetonitrile (30.0 mL), triphenylphosphine (808.0 mg, 3.08 mmol) and carbon tetrabromide (1021.0 mg, 3.08 mmol) were added under vigorous stirring at room temperature. After 2 hours the mixture was quenched with 15% NaOH and extracted with EtOAc (3×10 mL). The organic layers were dried and evaporated. The residue was chromatographed (10% methanol in chloroform) to give 480.0 mg of the title compound (75% yield) as yellow solid.

¹H NMR, 300 MHz, (CDCl₃) δ 1.66 (m, 2H), 1.77 (m, 2H), 3.26 (m, 2H), 3.36 (m, 2H), 7.22 (dd, 1H, J₁=8.2 Hz, J₂=4.4 Hz), 7.79 (br s, 1H), 7.88 (m, 2H), 7.97 (dd, 1H, J₁=8.9 Hz, J₂=1.9 Hz), 8.17 (d, 1H, J =1.5 Hz), 8.75 (dd, 1H, J₁=4.3 Hz, J₂=1.6 Hz); ESI-MS m/z 637 [2M+Na]⁺, 330 [M+Na]⁺, 308 [M+H]⁺. Anal. (C₁₄H₁₅BrN₂O) C, H, N.

### Method A

Quinoline-6-carboxylic acid [4-(4-pyridin-2-yl-piperazin-1-yl)-butyl]-amide hydrochloric acid salt (Compound A1)

A mixture of 1-(2-pyridyl)piperazine (0.85 g, 5.21 mmol), N-(4-bromobutyl)-quinoline-6-carboxamide (2.0 g, 5.21 mmol), triethylamine (0.58 g, 5.73 mmol) and acetonitrile (75 ml) was stirred at reflux for 20 hours. The mixture was evaporated, water (50 ml) and extracted with dichloromethane (3×30 ml). The mixture was washed with water (50 ml), dried and evaporated. Chromatography on silica gel with dichloromethane:methanol and aqueous ammonia (6:1:1%) as solvent gave the title compound as free base. The oil was solved and stirred in ethylacetate (5 ml) and HCl in ethanol (0.7 ml, 1M) was added. The product was isolated by filtration. Yield 300 mg (14%).

Quinoline-6-carboxylic acid {4-[4-(6-methyl-pyridin-2-yl)-piperazin-1-yl]-butyl}-amide hydrochloric acid salt (Compound A2)

Was prepared according to method A from 1-(6-methylpirid-2-yl)piperazine and N-(4-bromobutyl)-quinoline-6-carboxamide. Yield 39%.

### Example 2

### Biological Activity

In vitro Binding Studies

The affinity of Compound A2 of the invention for the dopamine and serotonin receptor subtypes was determined using standard receptor binding assays accomplished by MDS Pharma Services using the assay conditions specified below.

### Dopamin D3 (MDS Catalog No. 219800)

Human recombinant CHO cells

Ligand=0.015 μM [³H]-Spiperone

### Serotonin (5-Hydroxytryptamine) 5-HT_{1A} (MDS Catalog No. 271110)

Human recombinant (CHO cells)

Ligand=0.0036 μM [³H] 8-OH-DPAT

### Serotonin (5-Hydroxytryptamine) 5-HT_{2A} (MDS Catalog No. 271650)

Human recombinant (CHO cells)

Ligand=0.02 μM [³H] Ketanserin

These studies indicate that the compound of the invention shows an interesting, potent combination of activities at the dopamine D3 and serotonin 5-HT1a and 5-HT2a receptors.

1-11. (canceled)

12. An aryl piperazine derivative represented by Formula I 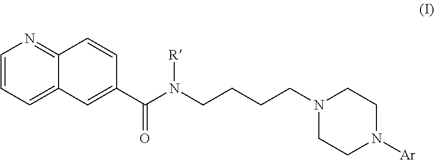
a stereoisomer thereof or a mixture of its stereoisomers, or a pharmaceutically acceptable salt thereof, wherein
R′ represents hydrogen or alkyl; and
Ar represents naphthyl or a heterocyclic, monocyclic or bicyclic aromatic group, which aromatic groups may optionally be substituted one or more times with substituents selected from alkyl, alkoxy, halo, trifluoromethyl, nitro and cyano.

13. The aryl piperazine derivative of claim 12, a stereoisomer thereof or a mixture of its stereoisomers, or a pharmaceutically acceptable salt thereof, wherein R′ represents hydrogen or alkyl.

14. The aryl piperazine derivative of claim 12, a stereoisomer thereof or a mixture of its stereoisomers, or a pharmaceutically acceptable salt thereof, wherein Ar represents naphthyl or a heterocyclic, monocyclic or bicyclic aromatic group, which aromatic groups may optionally be substituted one or more times with substituents selected from alkyl, alkoxy, halo, trifluoromethyl, nitro and cyano.

15. The aryl piperazine derivative of claim 12, which is
Quinoline-6-carboxylic acid [4-(4-pyridin-2-yl-piperazin-1-yl)-butyl]-amide; or
Quinoline-6-carboxylic acid {4-[4-(6-methyl-pyridin-2-yl)-piperazin-1-yl]-butyl}-amide;
a stereoisomer thereof or a mixture of its stereoisomers, or a pharmaceutically acceptable salt thereof.

16. A pharmaceutical composition comprising a therapeutically effective amount of an aryl piperazine derivative of claim 12, a stereoisomer thereof or a mixture of its stereoisomers, or a pharmaceutically acceptable addition salt thereof, together with at least one pharmaceutically acceptable carrier or diluent.

17. A method of diagnosis, treatment, prevention or alleviation of a disease or a disorder or a condition of a living animal body, including a human, which disorder, disease or condition is responsive to modulation of the dopamine and serotonin receptors, in particular the D₃, D₂-like and 5-HT₂ receptor subtypes, preferably the dopamine D₃ receptor subtype and/or the D₃/5-HT_{1A} or D₃/5-HT_{2A} receptor sybtypes, which method comprises the step of administering to such a living animal body in need thereof, a therapeutically effective amount of an aryl piperazine derivative according to claim 12, a stereoisomer thereof or a mixture of its stereoisomers, or a pharmaceutically acceptable salt thereof.

18. The method according to claim 17, wherein the disease or a disorder or a condition is a neurological or psychiatric disorders, in particular psychotic disorders, schizophrenia, depression, Parkinson's disease, Huntington's disease, movement disorders, dystonia, anxiety, restlessness, obsessive-compulsive disorders, mania, geriatric disorders, dementia, sexual dysfunction, musculo-skeletal pain symptoms, pain associated with fibromyalgia, sleep disorders, substance abuse or addiction, and abuse liability and withdrawal symptoms in drug addicts, cocaine abuse or addiction.

19. The method according to claim 17, wherein the disease or a disorder or a condition is a neurological or psychiatric disorder, in particular a psychotic disorder, preferably schizophrenia.
